(19) United States
(12) Patent Application Publication
(10) Pub. No.: US 2026/0199208 A1
(43) Pub. Date: Jul. 16, 2026

(54) Composition and process for treating keratin fibres using at least one particular pyridine derivative and at least one unsaturated fatty acid

(71) Applicant: L'OREAL, Paris (FR)

(72) Inventor: Andrew GREAVES, Aulnay-sous-Bois (FR)

(73) Assignee: L'OREAL, Paris (FR)

(21) Appl. No.: 19/135,646

(22) PCT Filed: Dec. 12, 2023
(86) PCT No.: PCT/EP2023/085406

§ 371 Date: Jun. 4, 2025

(30) Foreign Application Priority Data
Dec. 14, 2022 (FR) FR2213337

(51) Int. Cl. A61K 8/49; A61K 8/36; A61K 8/55; A61Q 5/12

(52) U.S. Cl. CPC A61K 8/4926; A61K 8/361; A61K 8/55; A61Q 5/12; A61K 2800/52

(57) ABSTRACT

The present invention relates to a composition comprising at least one particular pyridine derivative and at least one unsaturated fatty acid, and also to a process for treating keratin fibres using said composition.

### RELATED APPLICATIONS

The present application is a national stage application that claims priority to International Application No. PCT/EP2023/085406 filed Dec. 12, 2023, which claims priority to French Application No. 2213337 filed Dec. 14, 2022, the disclosures of which are incorporated herein by reference in their entirety.

### TECHNICAL FIELD OF THE INVENTION

The present invention relates to a composition comprising at least one particular pyridine derivative and at least one unsaturated fatty acid, and also to a process for treating keratin fibres using said composition.

### BACKGROUND OF THE INVENTION

The hair can be damaged and embrittled by external atmospheric agents such as pollution and bad weather, and also by mechanical or chemical treatments, or certain routines, such as brushing, combing, dyeing, bleaching, permanent-waving, relaxing and repeated washing. The hair may end up deformed and damaged and may in the long run become dry, coarse, brittle, dull, split and/or limp.

To overcome this, it is common practice to resort to hair compositions intended for conditioning the hair by giving it satisfactory cosmetic properties, notably smoothness, sheen, a soft feel, suppleness, lightness, good disentangling properties leading to easy combing, and good manageability of the hair which is thus easier to style and holds its shape. However, the conditioning effect obtained with these hair compositions fades rapidly over time, does not always show satisfactory persistence with respect to shampoo washing and does not prevent irreversible deformation of the hair due to elongation of the fibres, for example during combing or disentangling, nor does it repair or prevent hair breakage.

It is known practice to use pyridine derivatives such as pyridoxine or a salt thereof in hair compositions intended for conditioning the hair so as to preserve or even improve the quality of the hair. However, these derivatives may cause yellowing or browning of the hair compositions containing them during their storage. For this reason, the use of these derivatives in hair compositions has hitherto been relatively limited, notably preventing their use in high concentrations.

Thus, there is a real need to develop a hair composition characterized by good colour stability on storage, which comprises at least one particular pyridine derivative, making it possible to prevent irreversible deformation of the hair, notably of damaged and/or sensitized hair, due to elongation of the fibres, for example during combing or disentangling, and to repair or prevent breakage of the hair, notably of damaged and/or sensitized hair.

The Applicant has discovered, surprisingly, that all or some of these objectives can be achieved by means of the composition according to the present invention.

### SUMMARY OF THE INVENTION

According to a first aspect, a subject of the present invention is a composition comprising:
i) one or more pyridine derivatives chosen from the compounds of formula (I) below, salts thereof, isomers thereof, solvates thereof such as hydrates and mixtures thereof:

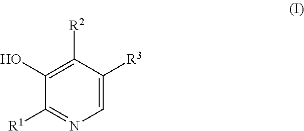

in which formula (I):
   R¹ represents a (C₁-C₄)alkyl group optionally substituted with one or more identical or different groups chosen from: hydroxyl (—OH) or amino (—NH₂);
   R² represents:
   a (C₁-C₄)alkyl group optionally substituted with one or more identical or different groups chosen from hydroxyl (—OH) or amino (—NH₂) and/or optionally interrupted with one or more oxygen, sulfur or nitrogen atoms, preferably oxygen; or
   a group (—CHO);
   R³ represents:
   a (C₁-C₄)alkyl group optionally substituted with one or more identical or different groups chosen from hydroxyl (—OH) or amino (—NH₂) or phosphate (—OP(O)(OH)₂) and/or optionally interrupted with one or more identical or different groups or heteroatoms chosen from O, S, N, —C(O)— and combinations thereof such as —SC(O)—, preferably chosen from O, or —SC(O)—;
ii) one or more fatty acids chosen from unsaturated fatty acids, salts thereof, isomers thereof, solvates thereof such as hydrates and mixtures thereof;
in which:
   the composition comprises a total content of compounds of formula (I), isomers thereof and mixtures thereof of more than 0.9% by weight relative to the total weight of the composition; and
   the mole ratio of the total amount of pyridine derivatives i) to the total amount of fatty acids ii) present in the composition is less than or equal to 1.15.

According to a second aspect, a subject of the present invention is a process for treating keratin fibres, comprising i) the application to the keratin fibres of a composition as defined previously.

According to a third aspect, a subject of the present invention is the use of a composition as defined previously for preventing irreversible deformation of keratin fibres during combing and/or disentangling.

According to a fourth aspect, a subject of the present invention is the use of a composition as defined previously for repairing and/or preventing keratin fibre breakage.

According to a fifth aspect, a subject of the present invention is the use of one or more fatty acids ii) as defined previously for stabilizing a composition comprising one or more pyridine derivatives i) as defined previously.

### DETAILED DESCRIPTION OF THE INVENTION

For the purposes of the present invention and unless otherwise indicated:
the term “keratin fibres” means fibres of human or animal origin, such as head hair, bodily hairs, the eyelashes, the eyebrows, wool, angora, cashmere or fur. According to the present invention, the keratin fibres are preferably human keratin fibres, more preferentially the head hair.
the term “damaged keratin fibres” means keratin fibres that are dry, rough, brittle, split and/or soft.
the term “sensitized keratin fibres” means keratin fibres that have been bleached, artificially coloured, relaxed and/or permed.
the term “fatty acid” means a linear or branched, saturated or unsaturated carboxylic acid comprising from 8 to 30 carbon atoms.
the term “unsaturated fatty acid” means a fatty acid comprising one or more ethylenic unsaturations in its structure, preferably one or two ethylenic unsaturations.
the term “alkyl group” means a saturated, linear or branched hydrocarbon-based radical comprising from 1 to 30 carbon atoms, preferentially from 1 to 26 carbon atoms, more preferentially from 1 to 22 carbon atoms, for example methyl, ethyl, n-propyl, isopropyl, butyl, n-pentyl, n-hexyl, n-decyl, n-undecyl, n-dodecyl, n-tetradecyl, n-hexadecyl or eicosyl.
the term “(Cₓ-C_{y})alkyl group” means an alkyl group comprising from x to y carbon atoms.
the term “alkenyl group” means a linear or branched hydrocarbon-based radical comprising one or more ethylenic unsaturations, preferably one or two ethylenic unsaturations, and from 2 to 40 carbon atoms, preferably from 2 to 26 carbon atoms, more preferentially from 2 to 24 carbon atoms, for example ethenyl, n-propenyl, isopropenyl, butenyl, n-pentenyl, n-hexenyl, n-decenyl, n-dodecenyl, tetradecenyl, hexadecenyl or eicosenyl.
the term “(Cₓ-C_{y})alkenyl group” means an alkenyl group comprising from x to y carbon atoms.
the term “salt” means an addition salt with an organic or mineral acid or base. The addition salts with an acid are notably chosen from addition salts with organic or mineral acids such as hydrochloric acid, hydrobromic acid, sulfuric acid, citric acid, succinic acid, tartaric acid, lactic acid, tosylic acid, benzenesulfonic acid, phosphoric acid or acetic acid. The addition salts with a base are notably chosen from the addition salts with organic or mineral bases such as alkali metal hydroxides, alkaline-earth metal hydroxides, ammonia, amines or alkanolamines.
the term “isomer” means an optical, geometrical or tautomeric isomer.

The terms “at least one” and “one or more” are synonymous and may be used interchangeably.

### Composition

According to a first aspect, a subject of the present invention is a composition as defined previously.

The Applicant has found, surprisingly, that the composition according to the present invention makes it possible to prevent irreversible deformation of the hair, notably of damaged and/or sensitized hair, due to elongation of the fibres, for example during combing or disentangling, and to repair or prevent breakage of the hair, notably of damaged and/or sensitized hair, while at the same time being stable and not undergoing any change in colour over time, notably during storage.

### Pyridine Derivatives

The composition comprises i) one or more pyridine derivatives chosen from the compounds of formula (I) as defined previously, salts thereof, isomers thereof, solvates thereof such as hydrates and mixtures thereof.

Preferably, the pyridine derivative(s) i) are chosen from compounds 1 to 10 below, salts thereof, isomers thereof, solvates thereof such as hydrates and mixtures thereof:

| | | |
|---|---|---|
| Compound 1 | 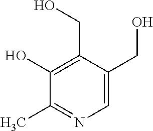 | Pyridoxine |
| Compound 2 | 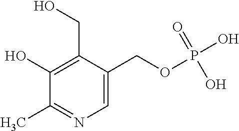 [SMILES: Cc1ncc(COP(=O)(O)O)c(CO)c1O] | Pyridoxine phosphate |
| Compound 3 | 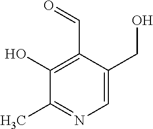 | Pyridoxal |
| Compound 4 | 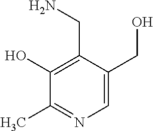 | Pyridoxamine |
| Compound 5 | 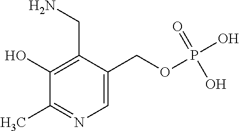 [SMILES: Cc1ncc(COP(=O)(O)O)c(CN)c1O] | Pyridoxamine phosphate |
| Compound 6 | 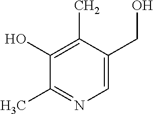 | 4-Deoxypyridoxine |
| Compound 7 | 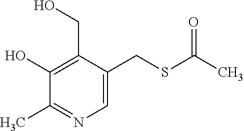 | Pyridoxine 6-thioacetate |
| Compound 8 | 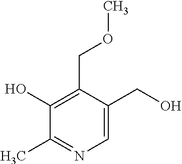 | 5-(Hydroxymethyl)-4- (methoxymethyl)-2- methylpyridin-3-ol |
| Compound 9 | 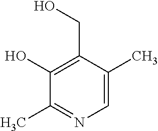 | 2,5-Dimethyl-3-hydroxy- 4-pyridinemethanol |
| Compound 10 | 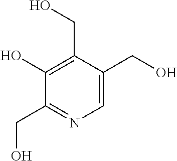 [SMILES: OCc1cnc(CO)c(O)c1CO] | 3-hydroxy=2,4,5- trihydroxymethylpyridine |

More preferentially, the pyridine derivative(s) i) are chosen from compound 1, salts thereof, solvates thereof such as hydrates and mixtures thereof.

Even more preferentially, the pyridine derivative(s) i) are chosen from pyridoxine, pyridoxine hydrochloride and mixtures thereof.

The composition comprises a total content of compounds of formula (I), isomers thereof and mixtures thereof of more than 0.9% by weight, preferably ranging from 1.1% to 10% by weight, more preferentially ranging from 1.5% to 9% by weight, even more preferentially ranging from 2% to 7% by weight relative to the total weight of the composition.

In the embodiments according to the invention in which one or more pyridine derivatives i) are in the form of a salt or solvate of a compound of formula (I), the term “total content of compounds of formula (I)” refers not to the salt or solvate content but to the content of compounds of formula (I). By way of example, when the composition according to the invention comprises only pyridoxine hydrochloride, the total content of compounds of formula (I) is not the pyridoxine hydrochloride content, but the pyridoxine content.

### Fatty Acids

The composition comprises ii) one or more fatty acids chosen from unsaturated fatty acids, salts thereof, isomers thereof, solvates thereof such as hydrates and mixtures thereof:

According to a preferred embodiment, the fatty acid(s) ii) are chosen from the compounds of formula (II) below, salts thereof, isomers thereof, solvates thereof such as hydrates and mixtures thereof:

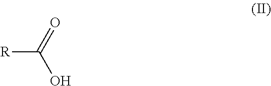

in which formula (II) R represents a (C₈-C₄₀)alkenyl group optionally substituted with one or more hydroxyl groups (—OH), preferably a (C₁₀-C₃₀)alkenyl group optionally substituted with one or more hydroxyl groups (—OH), more preferentially a (C₁₂-C₂₄)alkenyl group optionally substituted with one or more hydroxyl groups (—OH).

According to one particular embodiment, the fatty acid(s) ii) are chosen from the compounds of formula (II) as defined previously, salts thereof, isomers thereof, solvates thereof such as hydrates and mixtures thereof, in which formula (II) R represents an unsubstituted (C8-C40)alkenyl group, preferably an unsubstituted (C10-C30)alkenyl group, more preferentially an unsubstituted (C12-C24)alkenyl group.

According to another particular embodiment, the fatty acid(s) ii) are chosen from the compounds of formula (II) as defined previously, salts thereof, isomers thereof, solvates thereof such as hydrates and mixtures thereof, in which formula (II) R represents a (C8-C40)alkenyl group substituted with one or more hydroxyl groups (—OH), preferably a (C10-C30)alkenyl group substituted with one or more hydroxyl groups (—OH), more preferentially a (C12-C24)alkenyl group substituted with one or more hydroxyl groups (—OH).

According to another particular embodiment, the fatty acid(s) ii) are chosen from the compounds of formula (II) as defined previously, salts thereof, isomers thereof, solvates thereof such as hydrates and mixtures thereof, in which formula (II) R represents a (C8-C40)alkenyl group substituted with a hydroxyl group (—OH), preferably a (C10-C30)alkenyl group substituted with a hydroxyl group (—OH), more preferentially a (C12-C24)alkenyl group substituted with a hydroxyl group (—OH).

According to a particular embodiment, the fatty acid(s) ii) are chosen from monounsaturated fatty acids, salts thereof, isomers thereof, solvates thereof such as hydrates and mixtures thereof, preferably from myristoleic acid, palmitoleic acid, vaccenic acid, paullinic acid, oleic acid, elaidic acid, gadoleic acid, gondoic acid, erucic acid, nervonic acid, salts thereof, isomers thereof, solvates thereof such as hydrates and mixtures thereof.

According to a particular embodiment, the fatty acid(s) ii) are chosen from polyunsaturated fatty acids, salts thereof, isomers thereof, solvates thereof such as hydrates and mixtures thereof, preferably from linoleic acid, linolelaidic acid, eicosadienoic acid, docosadienoic acid, α-linolenic acid, γ-linolenic acid, dihomo-γ-linolenic acid, arachidonic acid, eicosapentaenoic acid, docosahexaenoic acid, ruminic acid, salts thereof, isomers thereof, solvates thereof such as hydrates and mixtures thereof.

According to a particular embodiment, the fatty acid(s) ii) are chosen from hydroxylated monounsaturated fatty acids, salts thereof, isomers thereof, solvates thereof such as hydrates and mixtures thereof, preferably from 10-hydroxy-cis-12-octadecenoic acid, 10-hydroxy-trans-11-octadecenoic acid, 12-hydroxy-cis-9-octadecenoic acid, 13-hydroxy-cis-9-octadecenoic acid, ricinoleic acid, isoricinoleic acid, salts thereof, isomers thereof, solvates thereof such as hydrates and mixtures thereof.

According to a particular embodiment, the fatty acid(s) ii) are chosen from hydroxylated polyunsaturated fatty acids, salts thereof, isomers thereof, solvates thereof such as hydrates and mixtures thereof, preferably from densipolic acid, auricolic acid, dimorphecolic acid, salts thereof, isomers thereof, solvates thereof such as hydrates and mixtures thereof.

According to a more preferred embodiment, the fatty acid(s) ii) are chosen from myristoleic acid, palmitoleic acid, vaccenic acid, paullinic acid, oleic acid, elaidic acid, gondoic acid, erucic acid, nervonic acid, linoleic acid, linolelaidic acid, eicosadienoic acid, docosadienoic acid, salts thereof, isomers thereof, solvates thereof such as hydrates and mixtures thereof.

According to an even more preferred embodiment, the fatty acid(s) ii) are chosen from oleic acid, salts thereof, solvates thereof such as hydrates and mixtures thereof, preferably oleic acid.

The fatty acid(s) ii) are present in the composition in a total content preferably ranging from 1.7% to 13% by weight, more preferentially ranging from 1.9% to 9% by weight, even more preferentially ranging from 2% to 6.5% by weight relative to the total weight of the composition.

The mole ratio of the total amount of pyridine derivatives i) to the total amount of fatty acids ii) present in the composition is less than or equal to 1.15, preferably ranging from 0.3 to 1.15, more preferentially ranging from 0.5 to 1.10.

According to a preferred embodiment, the composition according to the present invention comprises:
i) one or more pyridine derivatives chosen from pyridoxine, salts thereof, solvates thereof such as hydrates and mixtures thereof, preferably chosen from pyridoxine, pyridoxine hydrochloride and mixtures thereof;
ii) one or more fatty acids chosen from oleic acid, salts thereof, solvates thereof such as hydrates, and mixtures thereof, preferably oleic acid;
it being understood that:
   the composition comprises a total pyridoxine content of more than 0.9% by weight relative to the total weight of the composition; and
   the mole ratio of the total amount of pyridine derivatives i) to the total amount of fatty acids ii) present in the composition is less than or equal to 1.15.

### Other Characteristics of the Composition

The composition is a cosmetic composition, i.e. a composition which comprises a cosmetically acceptable medium, i.e. a medium that is compatible with human keratin fibres.

The composition may comprise a solvent chosen from water, ethanol, isopropanol, glycerol, propylene glycol, butylene glycol, pentylene glycol, hexylene glycol, dipropylene glycol, diethylene glycol, 2-butoxyethanol, propylene glycol monomethyl ether, diethylene glycol monoethyl ether, diethylene glycol monomethyl ether, and mixtures thereof.

The composition may comprise a solvent chosen from water, C₂-C₄ alcohols, polyols, polyol ethers, and mixtures thereof.

Preferably, the composition comprises a solvent chosen from water, ethanol, isopropanol, propylene glycol, and mixtures thereof.

More preferentially, the composition comprises a solvent chosen from water, ethanol, propylene glycol, and mixtures thereof.

The composition may also comprise at least one cosmetic ingredient chosen from nonionic, anionic, cationic and amphoteric surfactants, vitamins and provitamins other than vitamin B6, including panthenol, fillers, dyestuffs, nacreous agents, opacifiers, sequestrants, film-forming polymers, cationic, anionic or neutral polymers, associative polymers, plasticizers, silicones, thickeners, oils, antifoams, moisturizers, emollients, penetrants, fragrances, preserving agents, and mixtures thereof.

These usual cosmetic ingredients may be present in the composition in usual amounts, which may readily be determined by a person skilled in the art, and which may range, for each ingredient, from 0.01% to 20% by weight relative to the total weight of the composition. A person skilled in the art will take care to select the ingredients included in the composition, and also the amounts thereof, so that they do not harm the properties of the composition.

The composition may be in any presentation form conventionally used for a haircare application. In a non-limiting manner, the composition may be in the form of a lotion, a cream, a mousse, a gel, a spray or a lacquer.

The composition may be used in a rinse-off or leave-on application.

The composition may be in the form of a shampoo, a mask, a conditioning composition or a pre-shampoo. The composition may also be in the form of a composition to be added to or mixed with, before application, a shampoo, a mask or a conditioning composition.

The composition may be packaged in a pump bottle or in an aerosol container, so as to apply the composition in vapourized (lacquer) or foam form. In these cases, the composition preferably comprises at least one propellant.

The pH of the composition is preferably from 3 to 10, more preferentially from 3 to 7 and even more preferentially from 3.5 to 5. Preferably, the pH of the composition is equal to 4. The pH may be adjusted using an organic or mineral acid or an organic or mineral base usually used in cosmetics.

According to a preferred embodiment, the composition comprises a total content of colouring agents of less than 0.1% by weight, preferably less than 0.01% by weight, more preferentially less than 0.001% by weight relative to the total weight of the composition; even more preferentially, the composition is free of colouring agents.

The term “colouring agent” means an oxidation dye, a direct dye or a pigment.

The term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.

The term “direct dye” means a natural and/or synthetic dye, including in the form of an extract or extracts, other than oxidation dyes. These are coloured compounds that will spread superficially on the fibre. They may be ionic or nonionic, i.e. anionic, cationic, neutral or nonionic.

### Process for Treating Keratin Fibres

According to a second aspect, a subject of the present invention is a process for treating keratin fibres, comprising i) the application to the keratin fibres of a composition as defined previously.

The composition may be applied to dry or wet keratin fibres, preferably wet keratin fibres.

Preferably, the keratin fibres are damaged and/or sensitized keratin fibres, in particular damaged and/or sensitized hair.

The bath ratio of the composition applied to the keratin fibres may range from 0.1 to 10. By way of example, the bath ratio may be 0.4. The term “bath ratio” means the ratio between the total weight of the composition applied and the total weight of keratin fibres to be treated.

The process may comprise at least one additional step following step i), chosen from steps ii) to iv) below:
ii) a step of applying the composition to the keratin fibres, preferably for a period of at least 10 seconds, said application step optionally being performed under an airtight film of wrapper or plastic film type;
iii) a step of rinsing and/or washing the keratin fibres;
iv) a step of drying the keratin fibres in ambient air or using a heating device.

Preferably, the process comprises the additional steps ii) and iv) as described previously and performed in that order.

More preferentially, the process may comprise all of the additional steps ii), iii) and iv) as described previously and performed in that order.

The step of applying the composition may last from 10 seconds to 60 minutes, preferably from 30 seconds to 30 minutes.

The washing step may be performed, for example, using a shampoo.

The temperature of the heating device may range from 45° C. to 230° C., preferably from 45° C. to 100° C., more preferentially from 50° C. to 80° C. A hairdryer, a heating hood, an iron or a heating brush may be used, for example, as heating device.

The drying step iv) may also be a combination of drying with air or with a heating device at a temperature ranging from 50° C. to 80° C., such as a hairdryer or a heating hood, possibly followed by an ironing step, preferably with a straightening iron.

Steps i) to iii) or steps i) to iv) may be repeated as many times as required and each cycle of steps i) to iii) or i) to iv) may be spaced from the next by a few minutes to a few days or weeks. Different hair treatments of the invention may be performed between different application cycles, before or after.

In the case where the process does not comprise a step iii) of rinsing and/or washing the keratin fibres, the composition may, for example, comprise a solvent mixture such as a water/ethanol mixture which comprises at least 10%, preferably at least 20% and more preferentially at least 30% by weight of ethanol. The use of such a mixture can notably facilitate the evaporation of the composition.

### Uses

According to a third aspect, a subject of the present invention is the use of a composition as defined previously for preventing irreversible deformation of keratin fibres during combing and disentangling.

Preferably, the keratin fibres are damaged and/or sensitized keratin fibres, in particular damaged and/or sensitized hair.

According to a fourth aspect, a subject of the present invention is the use of a composition as defined previously for repairing and/or preventing keratin fibre breakage.

Preferably, the keratin fibres are damaged and/or sensitized keratin fibres, in particular damaged and/or sensitized hair.

According to a fifth aspect, a subject of the present invention is the use of one or more unsaturated fatty acids ii) as defined previously for stabilizing a composition, preferably for stabilizing the colour of a composition comprising one or more pyridine derivatives as defined previously.

### EXAMPLES

The following examples provide a better understanding of the invention, without, however, being limiting in nature.

a. Preparation of the Formulations

Two types of compositions Ax and By were prepared, in which the amount of pyridoxine HCl ranged from 1% to 5% by weight and the amount of oleic acid ranged from 0 to 8% by weight. Some of these compositions were evaluated as regards their stability and/or resistance to breakage and/or to irreversible deformation.

TABLE 1
Compositions Aₓ

| Ingredients | Mass % |
|---|---|
| Cetearyl alcohol | 7 |
| Mixture of myristyl stearate and myristyl palmitate | 2 |
| Behentrimonium chloride | 3.75 |
| Amodimethicone (and) trideceth-6 (and) cetrimonium | 8.5 |
| chloride | |
| Pyridoxine HCl | 1 to 5 |
| Oleic acid | 0 to 8 |
| Preserving agents | 0.43 |
| 5N NaOH | qs for pH 4.0 |
| Water | qs 100 |

TABLE 2
Compositions B_{y}

| Ingredients | Mass % |
|---|---|
| Ethanol | 30 |
| 1,2-Propylene glycol | 20 |
| Pyridoxine HCl | 1 to 5 |
| Oleic acid | 0 to 8 |
| 5N NaOH | qs for pH 4.0 |
| Water | qs 100 |

b. Study of the Stability of the Compositions

Some of the compositions Ax and By were placed in flasks and then placed in an oven at 66° C. for one week to study the change in colour during storage. The intensity of the change in colour of the compositions (yellowing/browning) after storage was evaluated using the scale: 0=no difference, 1=slight, 2=intense, 3=very intense. Only formulations with a score of 0 were judged to be colour-stable.

c. Study of Breakage and Irreversible Deformation of the Hair

Some of the compositions Ax and By were applied according to the application protocol described hereinbelow and evaluated with regard to their integrity, i.e. their resistance to breakage and irreversible deformation according to the test protocol described hereinbelow.

### Application Protocol

Locks of Caucasian hair, damaged by two successive bleaching operations, weighing 2.7 g and 27 cm long, are washed with DOP shampoo, and then squeezed dry between the fingers (two passes). The damp locks are then placed on a flat surface and compositions Ax or By are applied to said locks with a bath ratio of 0.4 (i.e. 0.4 g of composition per 1 g of hair).

The hair is then massaged with the fingers using circular movements throughout each lock (five times), and the locks are then left for 5 minutes. The locks are then rinsed with tap water for 10 seconds, squeezed dry and then dried at 60° C. (10 min/g of hair). These various steps are repeated for a total of five applications.

A lock of damaged and untreated hair is used as a reference.

### Test Protocol

The test consists in pulling on the damp hair to see if it breaks, deforms irreversibly or becomes deformed and then returns to its original shape and length. The test is commonly used by hairdressers to evaluate the integrity of their clients' hair. The test consists in taking a lock of hair, soaking it in water for 30 seconds and then taking and holding three hair fibres between the index finger and thumb of both hands (each hand takes one end of the fibres). The fibres are then put under slight tension by moving the fingers and thumbs of each hand apart so that there are 3 centimetres of fibre between the hands, and then pulling so that the fibres are deformed by a further 1 cm. A ruler is used to ensure that the stretching distances are respected. The fibres are held for 3 seconds and then the tension is gently released. The fibres are placed horizontally on absorbent paper for 5 minutes to allow them to dry, and are then evaluated.

The test is repeated five times, for a total of 15 fibres.

The hair integrity performance is graded according to the following rules:
If the sum of the broken and irreversibly deformed fibres is greater than or equal to 10, the hair has very poor integrity.
If the sum of the broken and irreversibly deformed fibres is between 6 and 9 inclusive, the hair has poor integrity.
If the sum of the broken and irreversibly deformed fibres is between 2 and 5 inclusive, the hair has good integrity.
If the sum of the broken and irreversibly deformed fibres is 1 or less, the hair has very good integrity.
   d. Results of the Stability Measurements:

The results of the stability measurements for each of the compositions tested are summarized in the table below.

TABLE 3

| | | | Mass % | | | |
|---|---|---|---|---|---|---|
| | | | pyridoxine | Mass | Pyridoxine | Intensity |
| | | | HCl (mass | % | HCl/oleic | of |
| Composi- | | Type of | % | oleic | acid mole | colour |
| tions | | composition | pyridoxine) | acid | ratio | change |
| A₁ | B₁ | Comparative | 1 (0.8) | 0 | N/A | 1 |
| A₂ | B₂ | Comparative | 2 (1.6) | 0 | N/A | 2 |
| A₃ | B₃ | Comparative | 3 (2.5) | 0 | N/A | 2 |
| A₄ | B₄ | Comparative | 4 (3.3) | 0 | N/A | 3 |
| A₅ | B₅ | Comparative | 5 (4.1) | 0 | N/A | 3 |
| A₆ | B₆ | Comparative | 1 (0.8) | 1 | 1.37 | 1 |
| A₇ | B₇ | Comparative | 1 (0.8) | 2 | 0.69 | 0 |
| A₈ | B₈ | Comparative | 1 (0.8) | 3 | 0.46 | 0 |
| A₉ | B₉ | Comparative | 1 (0.8) | 4 | 0.34 | 0 |
| A₁₀ | B₁₀ | Comparative | 1 (0.8) | 5 | 0.27 | 0 |
| A₁₁ | B₁₁ | Comparative | 1 (0.8) | 6 | 0.23 | 0 |
| A₁₂ | B₁₂ | Comparative | 1 (0.8) | 7 | 0.20 | 0 |
| A₁₃ | B₁₃ | Comparative | 1 (0.8) | 8 | 0.17 | 0 |
| A₁₄ | B₁₄ | Comparative | 2 (1.6) | 1 | 2.75 | 2 |
| A₁₅ | B₁₅ | Comparative | 2 (1.6) | 2 | 1.37 | 1 |
| A₁₆ | B₁₆ | Invention | 2 (1.6) | 3 | 0.92 | 0 |
| A₁₇ | B₁₇ | Invention | 2 (1.6) | 4 | 0.69 | 0 |
| A₁₈ | B₁₈ | Invention | 2 (1.6) | 5 | 0.55 | 0 |
| A₁₉ | B₁₉ | Invention | 2 (1.6) | 6 | 0.46 | 0 |
| A₂₀ | B₂₀ | Invention | 2 (1.6) | 7 | 0.39 | 0 |
| A₂₁ | B₂₁ | Invention | 2 (1.6) | 8 | 0.34 | 0 |
| A₂₂ | B₂₂ | Comparative | 3 (2.5) | 1 | 4.12 | 2 |
| A₂₃ | B₂₃ | Comparative | 3 (2.5) | 2 | 2.06 | 2 |
| A₂₄ | B₂₄ | Comparative | 3 (2.5) | 3 | 1.37 | 1 |
| A₂₅ | B₂₅ | Invention | 3 (2.5) | 4 | 1.03 | 0 |
| A₂₆ | B₂₆ | Invention | 3 (2.5) | 5 | 0.82 | 0 |
| A₂₇ | B₂₇ | Invention | 3 (2.5) | 6 | 0.69 | 0 |
| A₂₈ | B₂₈ | Invention | 3 (2.5) | 7 | 0.59 | 0 |
| A₂₉ | B₂₉ | Invention | 3 (2.5) | 8 | 0.52 | 0 |
| A₃₀ | B₃₀ | Comparative | 4 (3.3) | 1 | 5.49 | 3 |
| A₃₁ | B₃₁ | Comparative | 4 (3.3) | 2 | 2.75 | 3 |
| A₃₂ | B₃₂ | Comparative | 4 (3.3) | 3 | 1.83 | 2 |
| A₃₃ | B₃₃ | Comparative | 4 (3.3) | 4 | 1.37 | 1 |
| A₃₄ | B₃₄ | Invention | 4 (3.3) | 5 | 1.10 | 0 |
| A₃₅ | B₃₅ | Comparative | 5 (4.1) | 1 | 6.87 | 3 |
| A₃₆ | B₃₆ | Comparative | 5 (4.1) | 2 | 3.43 | 3 |
| A₃₇ | B₃₇ | Comparative | 5 (4.1) | 3 | 2.29 | 3 |
| A₃₈ | B₃₈ | Comparative | 5 (4.1) | 4 | 1.72 | 2 |
| A₃₉ | B₃₉ | Comparative | 5 (4.1) | 5 | 1.37 | 1 |
| A₄₀ | B₄₀ | Invention | 5 (4.1) | 6 | 1.14 | 0 |
| A₄₁ | B₄₁ | Comparative | 1 (0.8) | 1.1 | 1.25 | 1 |
| A₄₂ | B₄₂ | Comparative | 1 (0.8) | 1.25 | 1.10 | 0 |
| A₄₃ | B₄₃ | Comparative | 1 (0.8) | 1.5 | 0.92 | 0 |
| A₄₄ | B₄₄ | Comparative | 1 (0.8) | 1.75 | 0.78 | 0 |
| A₄₅ | B₄₅ | Comparative | 2 (1.6) | 2.1 | 1.31 | 1 |
| A₄₆ | B₄₆ | Comparative | 2 (1.6) | 2.25 | 1.22 | 1 |
| A₄₇ | B₄₇ | Invention | 2 (1.6) | 2.5 | 1.10 | 0 |
| A₄₈ | B₄₈ | Invention | 2 (1.6) | 2.75 | 1.00 | 0 |
| A₄₉ | B₄₉ | Comparative | 3 (2.5) | 3.1 | 1.33 | 1 |
| A₅₀ | B₅₀ | Comparative | 3 (2.5) | 3.25 | 1.27 | 1 |
| A₅₁ | B₅₁ | Comparative | 3 (2.5) | 3.5 | 1.18 | 1 |
| A₅₂ | B₅₂ | Invention | 3 (2.5) | 3.75 | 1.10 | 0 |
| A₅₃ | B₅₃ | Comparative | 4 (3.3) | 4.1 | 1.34 | 1 |
| A₅₄ | B₅₄ | Comparative | 4 (3.3) | 4.25 | 1.29 | 1 |
| A₅₅ | B₅₅ | Comparative | 4 (3.3) | 4.5 | 1.22 | 1 |
| A₅₆ | B₅₆ | Comparative | 4 (3.3) | 4.75 | 1.16 | 1 |
| A₅₇ | B₅₇ | Comparative | 5 (4.1) | 5.1 | 1.35 | 1 |
| A₅₈ | B₅₈ | Comparative | 5 (4.1) | 5.25 | 1.31 | 1 |
| A₅₉ | B₅₉ | Comparative | 5 (4.1) | 5.5 | 1.25 | 1 |
| A₆₀ | B₆₀ | Comparative | 5 (4.1) | 5.75 | 1.19 | 1 |

The results show that the compositions according to the present invention are stable.

e. Results of the Integrity Measurements

The results of the integrity measurements for each of the compositions tested are summarized in the table below.

TABLE 4

| | | Mass % | | Pyridoxine | | Number of |
|---|---|---|---|---|---|---|
| | | pyridoxine | | HCl/oleic | Number of | irreversibly |
| | Type of | HCl (mass % | Mass % | acid mole | broken | deformed |
| Composition | composition | pyridoxine) | oleic acid | ratio | fibres | fibres |
| Damaged and | — | — | — | — | 1 | 7 |
| untreated hair | | | | | | |
| B₄₂ | Comparative | 1 (0.8) | 1.25 | 1.10 | 1 | 6 |
| B₆₁ | Comparative | 2.25 (1.9) | 0 | N/A | 0 | 8 |
| B₆₂ | Comparative | 0 (0) | 2.25 | 0 | 0 | 7 |
| B₄₇ | Invention | 2 (1.6) | 2.5 | 1.10 | 0 | 2 |
| B₆₃ | Comparative | 4.5 (3.7) | 0 | N/A | 0 | 6 |
| B₆₄ | Comparative | 0 (0) | 4.5 | 0 | 0 | 6 |
| B₅₂ | Invention | 3 (2.5) | 3.75 | 1.10 | 0 | 3 |
| B₆₅ | Comparative | 6.75 (5.6) | 0 | N/A | 0 | 6 |
| B₆₆ | Comparative | 0 (0) | 6.75 | 0 | 0 | 7 |
| B₃₄ | Invention | 4 (3.3) | 5 | 1.10 | 0 | 2 |
| B₆₇ | Comparative | 9 (7.4) | 0 | N/A | 0 | 6 |
| B₆₈ | Comparative | 0 (0) | 9 | 0 | 0 | 7 |
| B₄₀ | Invention | 5 (4.1) | 6 | 1.14 | 0 | 0 |
| B₆₉ | Comparative | 11 (9) | 0 | N/A | 0 | 7 |
| B₇₀ | Comparative | 0 (0) | 11 | 0 | 0 | 6 |

The results show that the compositions according to the present invention make it possible to prevent irreversible deformation of the hair and hair breakage.

1. A composition comprising:
i) one or more pyridine derivatives chosen from the compounds of formula (I) below, salts thereof, isomers thereof, solvates thereof, hydrates thereof, or mixtures thereof: 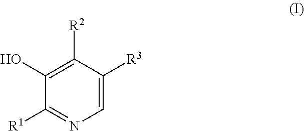
in which formula (I):
R¹ represents a (C₁-C₄)alkyl group optionally substituted with one or more identical or different groups chosen from: hydroxyl (—OH) or amino (—NH₂);
R² represents:
a (C₁-C₄)alkyl group optionally substituted with one or more identical or different groups chosen from hydroxyl (—OH) or amino (—NH₂) and optionally interrupted with one or more oxygen atoms, sulfur atoms, nitrogen atoms; or
a group (—CHO);
R³ represents:
a (C₁-C₄)alkyl group optionally substituted with one or more identical or different groups chosen from hydroxyl (—OH) or amino (—NH₂) or phosphate (—OP(O)(OH)₂) and optionally interrupted with one or more identical or different groups or heteroatoms chosen from O, S, N, —C(O)— and combinations thereof;
ii) one or more fatty acids chosen from unsaturated fatty acids, salts thereof, isomers thereof, solvates thereof, hydrates thereof, or mixtures thereof,
wherein
the composition comprises a total content of compounds of formula (I), isomers thereof or mixtures thereof of more than 0.9% by weight relative to the total weight of the composition; and
the mole ratio of the total amount of pyridine derivatives i) to the total amount of fatty acids ii) present in the composition is less than or equal to 1.15.

2. The composition according to claim 1, in which the pyridine derivative(s) i) are chosen from compounds 1 to 10 below, salts thereof, isomers thereof, solvates thereof, hydrates thereof, or mixtures thereof:

| | | |
|---|---|---|
| Compound 1 | 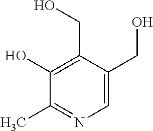 | Pyridoxine |
| Compound 2 | 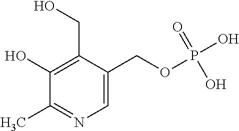 [SMILES: Cc1ncc(COP(=O)(O)O)c(CO)c1O] | Pyridoxine phosphate |
| Compound 3 | 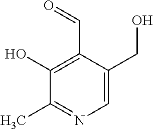 | Pyridoxal |
| Compound 4 | 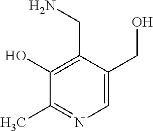 | Pyridoxamine |
| Compound 5 | 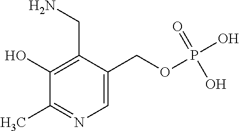 [SMILES: Cc1ncc(COP(=O)(O)O)c(CN)c1O] | Pyridoxamine phosphate |
| Compound 6 | 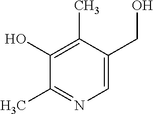 | 4-Deoxypyridoxine |
| Compound 7 | 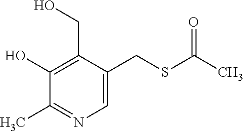 | Pyridoxine 6-thioacetate |
| Compound 8 | 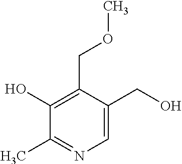 | 5-(Hydroxymethyl)-4- (methoxymethyl)-2- methylpyridin-3-ol |
| Compound 9 | 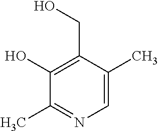 | 2,5-Dimethyl-3-hydroxy- 4-pyridinemethanol |
| Compound 10 | 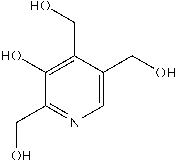 [SMILES: OCc1cnc(CO)c(O)c1CO] | 3-Hydroxy=2,4,5- trihydroxymethylpyridine |

3. The composition according to claim 1, in which the composition comprises a total content of compounds of formula (I), isomers thereof or mixtures thereof ranging from 1.1% to 10% by weight, by weight relative to the total weight of the composition.

4. The composition according to claim 1, in which the fatty acid(s) ii) are chosen from the compounds of formula (II) below, salts thereof, isomers thereof, solvates thereof, hydrates thereof, or mixtures thereof: 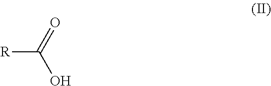
in which formula (II) R represents a (C₈-C₄₀)alkenyl group optionally substituted with one or more hydroxyl groups (—OH).

5. The composition according to claim 1, in which the fatty acid(s) ii) are chosen from myristoleic acid, palmitoleic acid, vaccenic acid, paullinic acid, oleic acid, elaidic acid and gondoic acid, erucic acid, nervonic acid, linoleic acid, linolelaidic acid, eicosadienoic acid, docosadienoic acid, salts thereof, isomers thereof, solvates thereof, hydrates thereof, or mixtures thereof.

6. The composition according to claim 1, in which the fatty acid(s) ii) are present in the composition in a total content ranging from 1.7% to 13% by weight, relative to the total weight of the composition.

7. The composition according to claim 1, in which the mole ratio of the total amount of pyridine derivatives i) to the total amount of fatty acids ii) present in the composition is from 0.3 to 1.15.

8. The composition according to claim 1, in which the pH of the composition is from 3 to 10.

9. The composition according to claim 1, in which the composition comprises a solvent chosen from water, C₂-C₄ alcohols, polyols, polyol ethers or mixtures thereof.

10. A process for treating keratin fibres, comprising i) the application to the keratin fibres of a composition as defined in claim 1.

11. The process according to claim 10, comprising at least one additional step following step i), chosen from steps ii) to iv) below:
ii) a step of applying the composition to the keratin fibres, preferably for a period of at least 10 seconds, said application step optionally being performed under airtight conditions;
iii) a step of rinsing, washing, or rinsing and washing the keratin fibres;
iv) a step of drying the keratin fibres in ambient air or using a heating device; or a combination of steps ii) through iv).

12. A method for treating keratin fibres with a composition according to claim 1, for preventing irreversible deformation of keratin fibres during combing and/or disentangling, or for repairing keratin fibres, or preventing the breakage of keratin fibres, or a combination thereof.

13. (canceled)

14. The method according to claim 12, in which the keratin fibres are damaged or are sensitized keratin fibres.

15. A method for stabilizing the colour of a composition comprising one or more pyridine derivatives i) as defined in claim 1, comprising se of one or more fatty acids ii) as defined in claim 1.

16. The method according to claim 15, in which the composition comprises a total content of compounds of formula (I), isomers thereof or mixtures thereof of more than 0.9% by weight relative to the total weight of the composition.
